(19) United States
(12) Patent Application Publication
(10) Pub. No.: US 2024/0241194 A1
(43) Pub. Date: Jul. 18, 2024

(54) PARTICLE THERAPY APPARATUS FOR IMAGING WITH MAGNETOMETERS

(71) Applicant: Elekta, Inc., Atlanta, GA (US)

(72) Inventor: Stuart Julian Swerdloff, Dunedin (NZ)

(21) Appl. No.: 18/559,973

(22) PCT Filed: May 13, 2022
(86) PCT No.: PCT/US2022/072314

§ 371 Date: Nov. 9, 2023

Related U.S. Application Data
(60) Provisional application No. 63/201,801, filed on May 13, 2021.

(51) Int. Cl. G01R 33/12; A61N 5/10

(52) U.S. Cl. CPC G01R 33/1276; A61N 5/1071; A61N 2005/1087

(57) ABSTRACT

Systems and techniques may be used for generating an image using one or more protons. For example, a technique may include detecting, over a time period using two orthogonal two-dimensional detector arrays, a magnetic field corresponding to a proton in motion. The technique may include determining a trajectory of the proton based on the magnetic field over the period of time, and generating a two-dimensional proton image using the trajectory. The two-dimensional proton image may be output for display.

### CLAIM OF PRIORITY

This application claims the benefit of priority to U.S. Provisional Application No. 63/201,801, filed May 13, 2021, titled “PARTICLE ARC APPARATUS FOR IMAGING WITH MAGNETOMETERS,” which is hereby incorporated herein by reference in its entirety.

### BACKGROUND

Radiation therapy (also referred to as radiotherapy) may be used in the treatment of cancer or other pathologies. Radiotherapy involves delivering a prescribed dose of radiation to a target region of a patient, for example, to a tumor or other cancerous tissue. The target region may be imaged prior to the administration of radiotherapy, and a treatment plan may be formulated based on, e.g., the size, location, and/or orientation of the target and the surrounding structures, among other things. A radiotherapy delivery device may then be used to deliver radiation in the form of one or more charged particle beams to the target region of the patient, in accordance with the treatment plan.

Accurate delivery of radiation to a patient promotes the safety and efficacy of radiotherapy treatment. Accordingly, prior to treatment, attenuation of a charged particle beam within the patient is predicted in order to determine where radiation will and will not be delivered to the body during treatment. Accurate determination of the location of the distal edge of a charged particle beam allows healthcare providers to assess how much dose was delivered to a patient and where the dose was delivered.

The intended location of the distal end of the beam and the dose delivered may be selected to ensure that surrounding healthy cells are not harmed or killed. If the charged particle beams delivered during radiotherapy have end points that are positioned in different locations than intended, then surrounding healthy structures may receive radiation instead of, or in addition to, the intended target region, and/or the target region may receive a different dose of radiation than intended. As a result, it is desirable to know the location of the actual, in vivo end point of a charged particle beam during treatment and to know how the actual location compared to the predicted end point location. Further, it may be desirable to assess the beam end point location during treatment so that the radiotherapy treatment may be altered or stopped if the actual, in vivo beam end point is not in the intended location.

Currently available technology for determining the actual, in vivo beam end point location of a charged particle beam may lack accuracy and may not provide information in a timely manner to be a useful assessment tool during treatment. For example, positron emission tomography (PET) may be used to determine tissue activation by a radiation particle beam in vivo. Yet, activation of the tissue may not provide a sufficiently accurate representation of the dose distribution of a charged particle beam or of the end terminus of the particle beam. Additionally, PET information may not be accessible in a timely manner. For example, it may take several minutes or more to acquire PET data.

### BRIEF DESCRIPTION OF THE DRAWINGS

FIG. 1 depicts an exemplary radiotherapy device, according to various embodiments of the present disclosure.

FIG. 2 depicts an exemplary system that may be used to provide particle beam end point detection, in accordance with embodiments of the present disclosure.

FIG. 3 depicts an exemplary system that may be used to provide particle beam end point detection, in accordance with embodiments of the present disclosure.

FIG. 4 depicts an exemplary system that may be used to provide particle beam end point detection, in accordance with embodiments of the present disclosure.

FIG. 5 illustrates a system including a particle depth detector, in accordance with an embodiment.

FIG. 6 illustrates a flowchart showing a technique for reconstructing a radiation dose deposited in a patient, in accordance with an embodiment.

FIG. 7 illustrates a flowchart showing a technique 700 for particle reconstruction, such as in proton therapy, in accordance with an embodiment.

FIG. 8 illustrates a system including a two orthogonal detector arrays, in accordance with an embodiment.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

The systems and techniques described herein may be used to reconstruct a radiation dose deposited in a patient more accurately and with less uncertainty. For example, an apparatus may use two sets of orthogonal detectors to determine a trajectory of a proton after exiting a patient. The orthogonal detectors may include a magnetometer to measure a strength or direction of a magnetic field, such as a magnetic field associated with the proton as it moves. Since the proton is a charged particle, it creates a magnetic field as it moves. The orthogonal detectors may determine a strength of the magnetic field at different points in time as the proton moves or stops to construct a trajectory of the proton. Using a known distance and orientation of the orthogonal detectors to a proton beam that emitted the proton or to a patient (e.g., to patient anatomy), a path through the patient may be reconstructed using the constructed trajectory.

The trajectory of the proton beamlet through the patient may be used to generate an image of the proton integral stopping power of the object the beamlet has passed through (e.g., the patient). In some examples, the image of the proton integral stopping power may be overlaid on an image of patient anatomy, such as a Digitally Reconstructed Radiograph created prior to the proton therapy. In other examples, the image of the proton integral stopping power may include 2D images, which may be used clinically in techniques analogous to uses of 2D radiographs. In other examples, where proton integral stopping power images have been obtained at a plurality of gantry angles, tomographic images of the proton stopping power can be reconstructed analogous to cone beam CT images and those images maybe overlaid on images of patient anatomy such as CT images captured prior to the proton therapy. The proton trajectory image generation may include identifying scatter (e.g., trajectories that may cause noise in an image) and remove the scatter when a proton is identified as moving in an abnormal or out of range trajectory.

In some examples, a material may be used to stop the proton. The material may include a cubic or other shaped container with a material such as a Poly(methyl methacrylate) (PMMA) (e.g., Lucite, Plexiglass, etc.) to stop the proton along its trajectory. The material selected may include a material that does not affect the magnetic field of the proton or one that affects the magnetic field of the proton in a known and predictable manner (e.g., dampening or magnifying (ferromagnetic)). As the proton is stopped in the material, an energy of the proton when it entered the material may be determined based on the time to stop, the distance traveled within the material, the trajectory, or the known properties of the material). The energy of the proton when it entered the material may be used to determine an energy the proton had when it exited a patient (e.g., optionally accounting for energy lost as the proton traveled through air from the patient to the material).

An apparatus may be used that includes the orthogonal detectors and optionally a material for stopping the proton. The apparatus may include a cubic container filled with PMMA or other material that stops proton travel. In an example, the distance the proton travels within the material (e.g., the depth the proton travels into the material along its trajectory) directly correlates to the energy of the proton. The distance travelled may depend on the stopping power of the material, which may be used to determine an energy of a proton when it entered the material.

### Exemplary Systems

Particle beam radiotherapy may be delivered in a number of different ways. For example, intensity modulated proton therapy (I MPT) may be used to deliver a narrow proton beam, commonly referred to as a ‘pencil beam’ or a ‘spot scan,’ to a target region via use of a magnetic field. The intensity of the beam and speed (i.e., energy) of the protons in each charged particle beam may be modulated in order to control the location and dose of radiation being delivered with each beam.

Charged particles for delivery may first be generated by an ion source. The type of ion source used may be specific to the type of charged particle being generated (e.g., protons, electrons, carbon ions, etc.). An accelerator may then be used to accelerate the charged particles to higher energies, e.g., above approximately 50 MeV. For example, energies may exceed 200 MeV, e.g., from approximately 230 to approximately 250 MeV or higher, and may exceed 400 MeV, e.g., 430 MeV. Exemplary accelerators may include cyclotrons, synchrotrons or synchrocyclotrons, dielectric wall accelerators, fixed-field alternating gradient accelerators, or laser proton accelerators. Once the charged particles are energized, the accelerated particle beam may be transported via one or more conduits, shaping magnets, and/or focusing magnets. In some embodiments, devices, e.g., range shifters, ion range compensators, and/or wedges, may be used to decrease particle speed (i.e., particle energy).

A charged particle beam may be delivered to a patient using either a fixed-beam radiation delivery device or via a delivery device having a rotational gantry. A fixed beam may be delivered at a horizontal, vertical, or intermediate angle to a patient. With a gantry, the beams may be delivered up to 360 degrees around the patient. Either the beams may be delivered as the gantry rotates, or the gantry may be rotated to a specific angle relative to the patient, and then the beams may be delivered from that angle, or a combination of both. The particle beams may be delivered by active spreading, also referred to as beam scanning (e.g., narrow, spot, or pencil beam scanning). With passive spreading, the radiation delivery device may include one or more collimators or compensators configured to shape the particle beam. For example, the particle beams may be shaped according to the shape, volume, and/or orientation of the tumor. With beam scanning, the radiation delivery device may include one or more magnets to deflect and steer the particle beams.

In some embodiments, one or more components of the radiation therapy delivery device may be located remote from the room in which the patient is contained. In some embodiments, each of the components may be located within the same room in which the patient is contained. For example, in some embodiments, the ion source and/or accelerator may be located remote from the patient, and one or more conduits, shaping magnets, focusing magnets, electronics, wedges, and/or other suitable devices may be used to transfer the particles from the accelerator to the radiation delivery device. In some exemplary arrangements, a single ion source and/or accelerator may supply multiple radiation therapy systems, which may each be located in different patient rooms or areas.

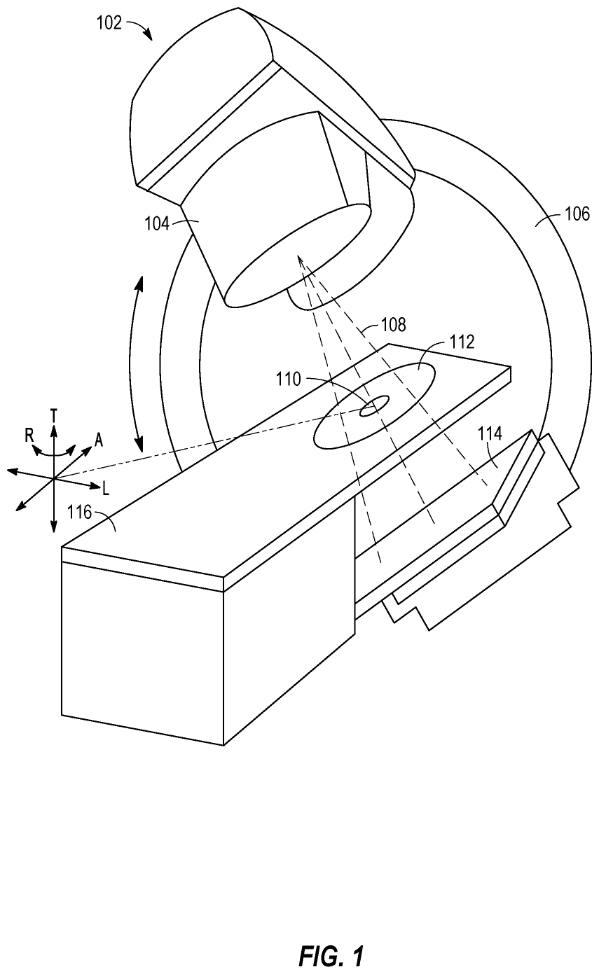

FIG. 1 depicts an exemplary particle beam radiation therapy system 102 configured to deliver one or more charged particle beams of radiation to a patient. In some aspects, radiation therapy system 102 may be configured to deliver a pencil beam of protons. A pencil beam may be delivered from radiation therapy system 102 using magnetic or electrostatic field controls (not shown) to generate a charged particle beam having a predetermined trajectory and a predetermined particle energy. Radiation therapy system 102 may be part of a larger imaging and radiotherapy system. For example, radiation therapy system 102 may operate independently or may operate in conjunction with an imaging acquisition system, for example, an MR imaging, X-ray imaging, CT imaging, ultrasound, or any other suitable medical imaging acquisition system. One or more components of an imaging system may acquire images before, during, and/or after radiotherapy treatment.

Radiation therapy system 102 may be used to provide real-time monitoring of the locations of the end points of emitted charged particle beams during radiotherapy, in accordance with various aspects of the disclosure. The systems may use information gleaned from detector array 114 of radiation therapy system 102 to track the locations of the end points of delivered particle beams in real time or to control or adapt a radiation therapy treatment plan in real time, as described further below.

System 102 may include a radiation therapy output 104 configured to deliver a charged particle beam of radiation 108 to a portion of a patient located in region 112. Radiation therapy output 104 may include one or more collimators, such as a multi-leaf collimator (MLC), or compensators. Collimators and/or compensators may be used to shape particle beam 108, e.g., based on the size and/or shape of the target region.

System 102 may also include a surface 116, for example, a table, bed, or couch, and a patient or a portion of a patient may be positioned on region 112 of surface 116 to receive a prescribed radiation therapy dose according to a radiation therapy treatment plan. In some embodiments, surface 116 may move relative to system 102. For example, surface 116 may move in a transverse (T) direction, a lateral direction (L), an axial direction (A), and/or may rotate about a transverse axis (R), e.g., to assist with moving the patient into and out of system 102, positioning the patient within system 102, setting up system 102, and/or cleaning or repairing system 102.

Radiation therapy output 104 may be coupled to a gantry 106 or other mechanical support and may be configured to move relative to the patient, relative to system 102, and/or relative to gantry 106. For example, radiation therapy output 104 may rotate on gantry 106 around an axis (A) extending through a central region of gantry 106. Radiation therapy output 104 may additionally or alternatively be moveable in a transverse direction or a lateral direction. This may, e.g., allow radiation therapy output 104 to be positioned relative to the patient. In some embodiments, radiation therapy system 102 may not include a gantry 106, and the location of radiation therapy output 104 may be fixed in place or may move less than 360 degrees around a patient positioned on surface 116.

One or more of surface 116, radiation therapy output 104, and/or gantry 106 may be manually or automatically positioned relative to one another in system 102. Characteristics of charged particle beam 108 output by radiation therapy output 104 may be manually or automatically controlled and may be determined according to a specified dose of radiation intended for a specific region of interest of the patient for a particular radiotherapy delivery session during a treatment plan. A sequence of radiation therapy deliveries may be specified according to a radiation therapy treatment plan, for example, one or more different orientations or locations of gantry 106, surface 116, or radiation therapy output 104 may be adjusted based on the sequence. For example, radiation therapy output 104 may move along gantry 106 around axis A and may output one or more particle beams 108 at a number of different locations. Thus, charged particle beams 108 from radiation therapy output 104 may be delivered to the target region from a number of different directions. In some embodiments, deliveries of radiation therapy from different angles may occur sequentially but each may end at region of interest 110. In this way, a prescribed cumulative dose of radiation therapy may be delivered to a target region within the patient from different angles. During delivery, exposure and damage to structures surrounding the target region may be reduced or avoided with precise delivery of radiation by, e.g., controlling the position of radiation therapy output 104, the energy of charged particle beams 108, and/or the intensity of the charged particle beams 108.

Detector array 114 may be mounted on gantry 106 approximately 180 degrees away from radiation therapy output 104 and may move with radiation therapy output 104 to maintain alignment to receive charged particle beams 108 as gantry 106 rotates. In some aspects, detector array 114 may be movably mounted (either automatically or manually movable) on radiotherapy system 102. For example, detector array 114 may be moved closer to or further from the patient to position detector array 114 relative to the patient once the patient is arranged on surface 116. If the orientation of radiation therapy output 104 is fixed, then detector array 114 may be fixed in position opposite the radiation therapy output 104 to receive a charged particle beam 108 delivered from the radiation therapy output 104. In some aspects, detector array 114 may be moved relative to radiation therapy output 104 or the patient.

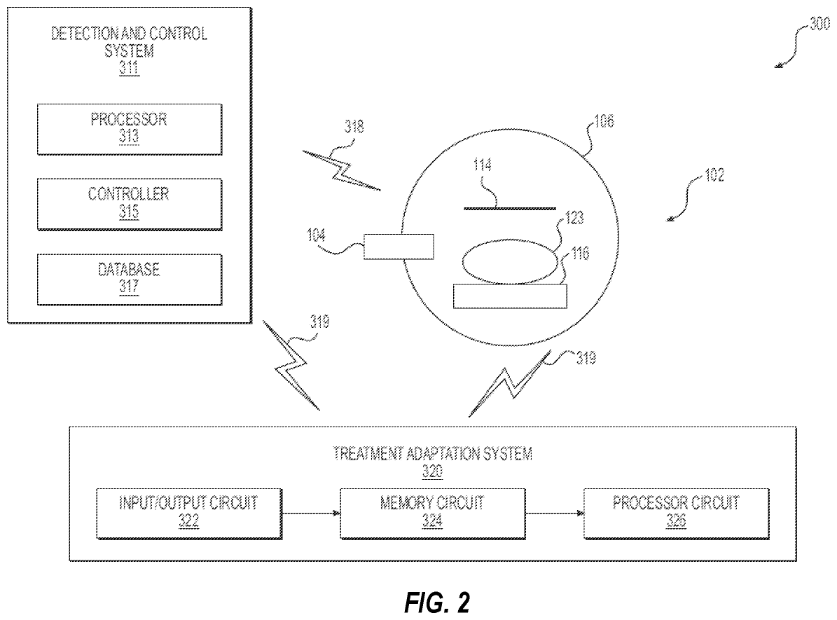

FIG. 2 depicts an exemplary radiotherapy control system 300 that may be used to provide real-time charged particle beam end point detection and feedback in accordance with various embodiments of the disclosure. Radiotherapy control system 300 may use measurements of the magnetic field detected by detector array 114 obtained in real time to track, control, and/or adapt a radiation therapy treatment plan during the administration of radiotherapy. Radiotherapy control system 300 may include radiotherapy system 102 of FIG. 1. Radiotherapy control system 300 may also include detection and control system 311, which may include processor 313. Processor 313 may be configured to process measurement data received from detector array 114, for example, to perform one or more calculations. In some embodiments, however, radiotherapy system 102 may incorporate a processor and may communicate processed information to detection and control system 311. In some embodiments, a separate processor 313 may be included in radiotherapy control system 300 instead of, or in addition to, a processor integrated into radiotherapy system 102. If a processor is only included in detection and control system 311, then raw measurements from detector array 114 may be communicated to processor 313. Radiotherapy system 102, including detector array 114, may be connected to detection and control system 311, as depicted by lightning bolt 318 (lightning bolt 318 may represent a wired or wireless connection).

Detection and control system 311 may further include a controller 315 in communication with system 102, as depicted by lightning bolt 318 (lightning bolt 318 may represent a wired or wireless connection). Detection and control system 311 may also include a database 317, for example, to store acquired magnetic field measurement information from detector array 114. Measurements received from detector array 114 and/or processed by processor 313 may be used to control and/or adapt treatment of a patient 123. Raw or processed measurement information from detector array 114 may be communicated to controller 315 and database 317 to adapt treatment of patient 123.

Processor 313 (and/or integrated processor within radiotherapy system 102) may acquire and process detected magnetic field information from detector array 114 as one or more charged particle beams are delivered to a patient located within radiotherapy system 102 in order to determine the locations of the end points of each charged particle beam delivered. This information may be compared to the expected locations of the beam end points determined during the treatment planning phase. In some embodiments, the expected location information may be stored in database 317. For example, during a treatment planning phase, a healthcare worker, e.g., physician, nurse, physicist, or technician, may acquire 3-D planning image data prior to treatment of the patient, e.g., via an imaging system separate from and/or integrated within radiotherapy system 102. The 3-D planning image data may be used to determine a precise location of a target region of the patient, e.g., a tumor. A stopping power map may be generated for the patient based on the patient anatomy and/or a model of the predicted magnetic field and possible beam end point depths may be generated. In some embodiments, this planning information may be received in database 317 and/or memory circuit 324. Based on the imaging—which may show the locations of different structures through which the charged particle beams may pass and/or surrounding structures—and based on the stopping power of these structures, the desired end points of the charged particle beams may be pre-calculated. The energy and intensity of the charged particle beams delivered during a treatment session may be determined in order to deliver radiation to the desired locations within the patient's body.

Controller 315 may control one or more aspects of system 300. For example, controller 315 may control portions of radiotherapy system 102. Controller 315 may control the position of the patient (e.g., by controlling movement of surface 116), may control the radiation dosage (e.g., energy and/or intensity) of charged particle beams emitted from radiation therapy output 104, may control or adapt a beam aperture shape or size (e.g., to track the target region), and/or may control the movement and/or positioning of radiation therapy output 104 and/or detector array 114 or the positions of individual detectors 119 relative to patient 123 or relative to each other (e.g., by controlling rotation around gantry 106 or other movements).

System 300 may include a treatment adaptation system (TAS) 320 in communication with detection and control system 311, as represented by lightning bolt 319 (which may represent a wired or wireless connection). TAS 320 may receive data from detection and control system 311 and/or radiotherapy system 102 regarding the magnetic field detected by detector array 114 and/or the position of the charged particle beam end point locations (collectively referred to as detection data). TAS 320 may include an input/output circuit 322 for receiving and transmitting data, a memory circuit 324 for buffering and/or storing data, and a processor circuit 326. Memory circuit 324, which may be any suitably organized data storage facility, may receive magnetic field detection data from detection and control system 311. Memory circuit 324 may receive the detection data via a wireless or wired connection or through conventional data ports and may include circuitry for receiving analog detection data and analog-to-digital conversion circuitry for digitizing the detection data. Memory circuit 324 may provide the detection data to processor circuit 326, which may implement the functionality of the present invention in hardware or software, or a combination of both, on a general- or special-purpose computer. In some embodiments, processor circuit 326 may be a graphical processing unit (GPU).

During operation, radiotherapy system 102 may deliver charged particle beams to a target region of a patient. Detector array 114 may measure the magnetic fields generated by charged particle beams, and the detected information may be processed or otherwise analyzed in order to determine the locations of the end points of the charged particle beams. The detector information collected may be stored in database 317, where other, prior detector information and/or beam end point information may also be stored (for example, the expected beam end point locations based on pre-planning, the beam end point locations from previously emitted charged particle beams delivered in the same treatment session and/or a previous treatment session), and this detector information may be raw or processed. Detector information may be communicated from detection and control system 311 to TAS via input/output circuit 322. The detector information may be stored in memory circuit 324 and communicated to processor circuit 326. Processor circuit 326 may be programmed to carry out a number of different processes and may have software loaded on it to perform different processes, including the calculation of beam end point locations (e.g., using best fit methods or other suitable algorithms and methods), analysis of beam end point locations in current or prior treatments, and/or the comparison of beam end point locations relative to the expected beam end point locations. The processed detector information may be stored in memory circuit 324 and/or may be communicated to detection and control system 311.

Memory circuit 324 may also store information regarding a treatment plan for patient 123, and this information may also be shared with processor circuit 326. Processor circuit 326 may compare real-time, processed detector information from radiotherapy system 102 and/or detection and control system 311 with the predetermined treatment plan for the patient to determine whether the radiotherapy being delivered to patient 123 matches the intended treatment plan for that radiotherapy session. If a variation is detected between the actual delivery of radiotherapy (determined using the detector information generated using detector array 114) and the treatment plan, and that variation falls outside of an allowable threshold of variation, then TAS 320 may communicate this to detection and control system 311. TAS 320 may modify the treatment plan or may stop the radiotherapy treatment altogether, for example, if the variation is beyond a threshold level. This modification or cessation may be communicated to controller 315 of detection and control system 311, which may control a portion of radiotherapy system 102. For example, controller 315 may alter a position of patient 123 via movement of surface 116, may alter the location, shape, energy, and/or intensity of charged particle beams output from radiation therapy output 104 to alter the location of the end points radiation therapy output 104, or may stop the output of charged particle beams from radiation therapy output 104. In this way, detector information may be processed in real time and may be used to control the administration of radiotherapy in real time.

In some embodiments, in addition to or instead of modifying or stopping the current treatment session in real time, future treatment sessions may be modified. For example, if the actual location of one or more charged particle beam end points is different than the expected location, then characteristics (e.g., energy and/or intensity) of subsequent charged particle beams delivered in future treatment sessions may be altered in order to compensate for the deviation and to make sure that the intended dose and intended location of the dose of radiation delivered to the target region is achieved over the course of the treatment sessions.

It should be noted that although a separate detection and control system 311 and a separate TAS 320 are depicted, the systems may be combined into one unit or may be distributed in any suitable manner across multiple separate units. Additionally, one or more units may be located within the treatment administration area or may be located remote from the treatment area. In some embodiments, the processing and data analysis may be integrated into radiotherapy system 102, may be performed within larger detection and radiotherapy system 300, or system 300 and/or system 102 may be connected to a network that is connected to the Internet, and a computer remote from radiotherapy system 102 may perform the processing and analyses described in embodiments of the present disclosure.

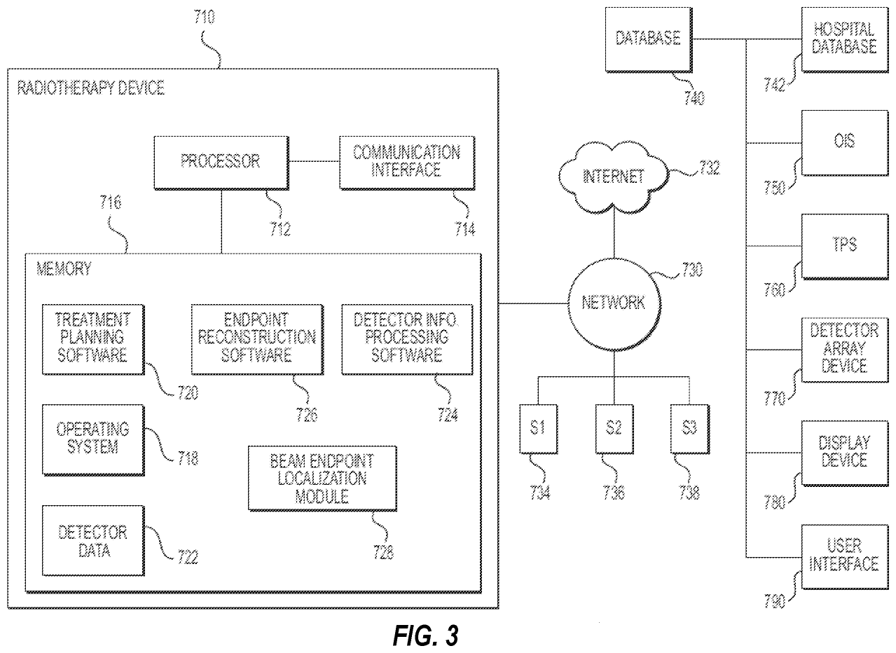

The processing of information measured by detector array 114 disclosed herein may be carried out on any suitable computer and/or medical system. FIG. 3 illustrates an exemplary radiotherapy system 700 for performing real-time charged particle beam end point localization and tracking during radiation therapy treatment using the novel techniques described above. Radiotherapy system 700 may include a radiation therapy device 710 connected to a network 730 that is connected to an Internet 732. Network 730 may connect radiation therapy device 710 with one or more of a database 740, a hospital database 742, an oncology information system (OIS) 750 (e.g., which may include patient information), a treatment planning system (TPS) 760 (e.g., for generating radiation therapy treatment plans to be carried out by the radiotherapy device 710), a detector array device 770, a display device 780, and/or a user interface 790. Each of these components may be housed in the same region as radiotherapy device 710 or may be remote from radiotherapy device 710, for example, connected to radiotherapy device 710 by the Internet or network connection.

Radiotherapy device 710 may include a processor 712, a memory device 716, and a communication interface 714. Memory device 716 may store computer executable instructions for one or more of an operating system 718, treatment planning software 720, detector information processing software 724, beam end point reconstruction software 726, a beam end point localization module 728, and/or any other computer executable instructions to be executed by processor 712. These executable instructions may configure processor 712 to execute the steps of the exemplary embodiments described above, including, e.g., calculation of beam end point locations, the calculation of a best fit depth for each beam end point location, a comparison of the actual or best fit beam end point locations relative to the expected beam end point locations, updating positions of the beam end points in the plan as-delivered, recalculating the dose distribution for the current or future fractions, updating the current or future treatment plan, and/or updating the stopping power map.

Processor 712 may be communicatively coupled to memory device 716, and processor 712 may be configured to execute computer executable instructions stored thereon. For example, processor 712 may execute detector information processing software 724 and/or end point reconstruction software 726 to implement functionalities of each and may combine these with the functionalities of beam end point localization module 728 in order to determine locations of the end points of a series of charged particle beams delivered to a patient during administration of radiotherapy. In addition, processor 712 may execute treatment planning software 720 (e.g., Monaco® software manufactured by Elekta) that may interface with detector processing software 724, end point reconstruction software 726, and/or beam end point localization module 728.

Processor 712 may be a processing device, include one or more general-purpose processing devices such as a microprocessor, central processing unit (CPU), graphics processing unit (GPU), an accelerated processing unit (APU), or other suitable equipment. In some embodiments, processor 712 may be a complex instruction set computing (CISC) microprocessor, reduced instruction set computing (RISC) microprocessor, very long instruction Word (VLIW) microprocessor, a processor implementing other instruction sets, or processors implementing a combination of instruction sets. Processor 712 may also be one or more special-purpose processing devices, such as an application specific integrated circuit (ASIC), a field programmable gate array (FPGA), a digital signal processor (DSP), a System on a Chip (SoC), or the like. As would be appreciated by those skilled in the art, in some embodiments, processor 712 may be a special-purpose processor, rather than a general-purpose processor, for example, one typically used for medical imaging and/or radiotherapy, and therefore may have one or more graphical processing units and accelerated processing units. Processor 712 may include one or more known processing devices, such as a microprocessor from the Pentium™, Core™, Xeon™, or Itanium® family manufactured by Intel™, the Turion™, Athlon™ Sempron™, Opteron™, FX™, Phenom™ family manufactured by AMD™ or any of various processors manufactured by Sun Microsystems, or other suitable processors. Processor 712 may also include graphical processing units, such as a GPU from the GeForce®, Quadra®, Tesla® family manufactured by Nvidia™, GMA, Iris™ family manufactured by Intel™, or the Radeon™ family manufactured by AMD™, or other suitable processors. Processor 712 may in some embodiments include accelerated processing units such as the Desktop A-4(6, 8) Series manufactured by AMD™ or the Xeon Phi™ family manufactured by Intel™. In one embodiment, processor 712 may be configured to process large amounts of data from detector array device 770 (which may be part of radiotherapy system 102) in real time, where “real time” means that the input data is processed at a speed that allows output or feedback to be made available during a radiotherapy procedure. The disclosed embodiments are not limited to any type of processor(s) otherwise configured to meet the computing demands of identifying, analyzing, maintaining, generating, and/or providing large amounts of detection and/or imaging data or manipulating such detection and/or imaging data to localize and track beam end point locations or to manipulate any other type of data consistent with the disclosed embodiments. In addition, the term “processor” may include more than one processor, for example, a multi-core design or a plurality of processors each having a multi-core design. Processor 712 may execute sequences of computer program instructions stored in memory 716 to perform the various operations, processes, and methods described above.

Memory device 716 may store detector data 722 received from detector array device 770. Memory device 716 may also store any other suitable type of data/information in any format that may be used by radiotherapy device 710 to perform operations consistent with the disclosed embodiments. Memory device 716 may include a read-only memory (ROM), a flash memory, a random access memory (RAM), a dynamic random access memory (DRAM), such as synchronous DRAM (SDRAM) or Rambus DRAM, a static memory (e.g., flash memory, static random access memory), etc., on which computer executable instructions may be stored in any format. In an exemplary embodiment, memory device 716 may be a plurality of memory devices. In some embodiments, memory device 716 may include a plurality of memory devices that are remotely located but accessible to processor 712. The computer program instructions may be accessed by processor 712, read from the ROM, or any other suitable memory location, and loaded into the RAM for execution by processor 712. For example, memory 716 may store one or more software applications. Software applications stored in memory 716 may include, for example, an operating system 718 for common computer systems, as well as for software-controlled devices. Further, memory 716 may store an entire software application or only a part of a software application that is executable by processor 712. For example, memory device 716 may store one or more radiation therapy treatment plans generated by treatment planning system 760 and/or may store treatment planning software 720.

In some embodiments, memory device 716 may include a machine-readable storage medium. Exemplary embodiments may include a single medium or may include multiple media (e.g., a centralized or distributed database, and/or associated caches and servers) that store the one or more sets of computer executable instructions or data. The term “machine-readable storage medium” refers to any medium that is capable of storing or encoding a set of instructions for execution by the machine and that cause the machine to perform any one or more of the methodologies of the present disclosure. The term “machine readable storage medium” shall accordingly be defined as including, but not be limited to, solid-state memories, optical and magnetic media, or the like. For example, memory 716 may be one or more volatile, non-transitory, or non-volatile tangible computer-readable media.

Radiotherapy device 710 may communicate with a network 730 via a communication interface 714, which may be communicatively coupled to processor 712 and memory 716. Communication interface 714 may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as a WiFi adaptor), a telecommunication adaptor (e.g., 3G, 4G/LTE and the like), or other suitable connections. Communication interface 714 may include one or more digital and/or analog communication devices that permit radiotherapy device 710 to communicate with other machines and devices, such as remotely located components, via a network 730.

Network 730 may provide the functionality of a local area network (LAN), a wireless network, a cloud computing environment (e.g., software as a service, platform as a service, infrastructure as a service, etc.), a client-server, a wide area network (WAN), or the like. Therefore, network 730 may allow data transmission between radiotherapy device 710 and a number of other devices, including TPS 760, OIS 750, and detector array device 770. Further, data generated by TPS 760, OIS 750, and detector array device 770 may be stored in memory 716, database 740, and/or hospital database 742. The data may be transmitted/received via network 730 and through communication interface 714 in order to be accessed by processor 712.

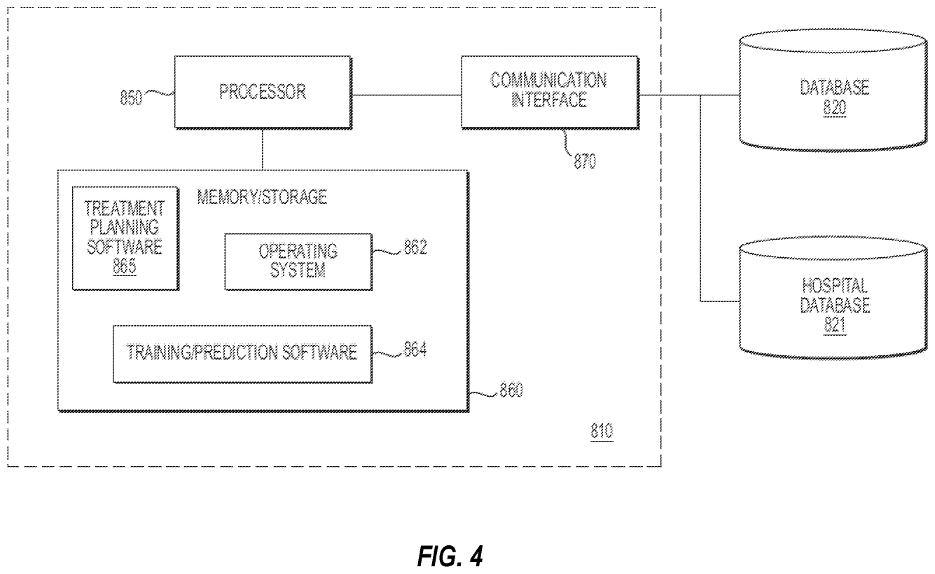

FIG. 4 depicts another exemplary data processing system that may be used to carry out the processing of information generated from detector array 114 and/or comparison of the detected charged particle beam end points relative to the expected end points. FIG. 4 illustrates an embodiment of data processing device 810 that is communicatively coupled to a database 820 and a hospital database 821. As shown in FIG. 4, data processing device 810 may include a processor 850, a memory or storage device 860, and a communication interface 870. Memory/storage device 860 may store computer executable instructions, such as an operating system 862, training/prediction software 864, treatment planning software 865, and any other computer executable instructions to be executed by the processor 850.

Processor 850 may be communicatively coupled to a memory/storage device 860 and configured to execute the computer executable instructions stored thereon. For example, processor 850 may execute training/prediction software 864 to implement functionalities of the embodiments described herein. In addition, processor device 850 may execute treatment planning software 865 (e.g., such as Monaco® software manufactured by Elekta) that may interface with training/prediction software 864.

Processor 850 may communicate with database 820 through communication interface 870 to send/receive data to/from database 820. One skilled in the art would appreciate that database 820 may include a plurality of devices located either in a central or distributed manner. In addition, processor 850 may communicate with the hospital database 821 to implement functionalities of radiation therapy system 102, as shown in FIG. 1.

Processor 850 may be a processing device and may include one or more general-purpose processing devices such as a microprocessor, central processing unit (CPU), graphics processing unit (GPU), or the like. More particularly, processor device 850 may be a complex instruction set computing (CISC) microprocessor, reduced instruction set computing (RISC) microprocessor, very long instruction Word (VLIW) microprocessor, a processor implementing other instruction sets, or processors implementing a combination of instruction sets. Processor 850 may also be one or more special-purpose processing devices such as an application specific integrated circuit (ASIC), a field programmable gate array (FPGA), a digital signal processor (DSP), a System on a Chip (SoC), or the like. As would be appreciated by those skilled in the art, in some embodiments, processor 850 may be a special-purpose processor, rather than a general-purpose processor.

Memory/storage device 860 may include a read-only memory (ROM), a flash memory, a random access memory (RAM), a static memory, etc. In some embodiments, memory/storage device 860 may include a machine-readable storage medium. While the machine-readable storage medium in an embodiment may be a single medium, the term “machine-readable storage medium” should be taken to include a single medium or multiple media (e.g., a centralized or distributed database, and/or associated caches and servers) that store the one or more sets of computer executable instructions or data. The term “machine-readable storage medium” shall also be taken to include any medium that is capable of storing or encoding a set of instructions for execution by the machine and that cause the machine to perform any one or more of the methodologies of the present disclosure. The term “machine readable storage medium” shall accordingly be taken to include, but not be limited to, solid-state memories, optical, and magnetic media.

Communication interface 870 may include a network adaptor, a cable connector, a serial connector, a USB connector, a parallel connector, a high-speed data transmission adaptor such as fiber, USB 3.0, thunderbolt, a wireless network adaptor such as a WiFi adaptor, or a telecommunication (3G, 4G/LTE and the like) adaptor. The communication interface 870 may provide the functionality of a local area network (LAN), a wireless network, a cloud computing environment (e.g., software as a service, platform as a service, infrastructure as a service), a client-server, or a wide area network (WAN). Processor 850 may communicate with database 820 or other devices or systems via communication interface 870.

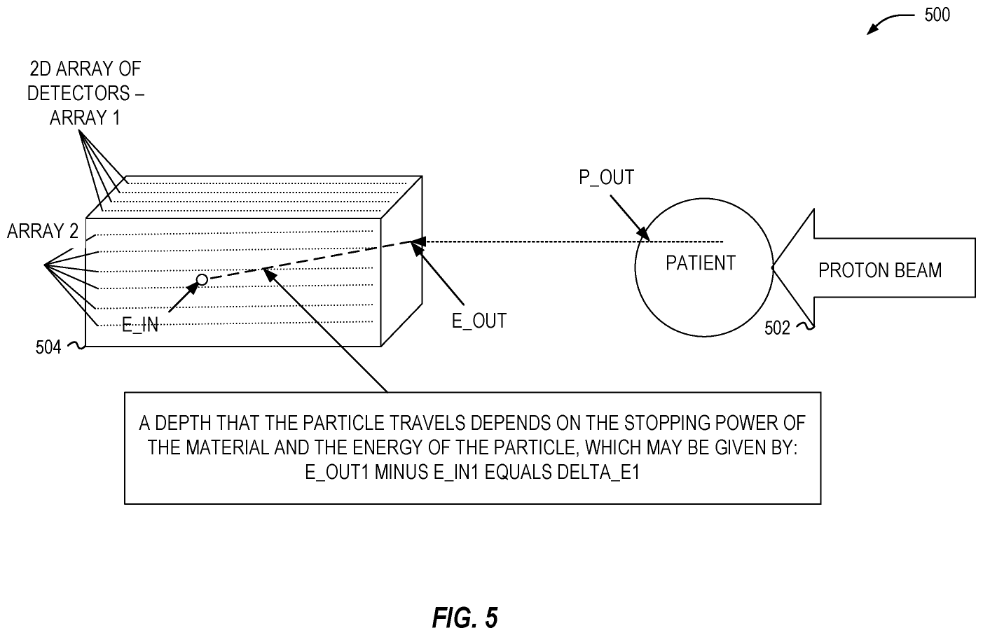

FIG. 5 illustrates a system 500 including a particle depth detector, in accordance with an embodiment. The system 500 includes a proton beam 502 that delivers a proton to a patient. The proton fully penetrates the patient and exits the patient. A detector apparatus 504 receives the proton, which enters the apparatus at a particular point and has its speed slowed and eventually stopped by a material of the detector apparatus 504 (e.g., Poly(methyl methacrylate) (PMMA), which may Plexiglass, Lucite, etc.). The detector apparatus 504 includes two orthogonal 2D arrays of detectors, such as magnetometers. The detector apparatus 504 may be used to determine a trajectory of the proton through the patient. For example, by determining the entry location to the detector apparatus 504, as well as the end point of the proton within the detector apparatus 504, the trajectory may be determined (e.g., by a processor). This trajectory may be applied to a known position and orientation of the patient during the procedure, such that the trajectory through the patient may be output. This trajectory through the patient may be displayed, such as by being output as a proton image, overlaid on a cone beam CT, or the like. In some example, the proton may be used therapeutically to treat the patient as well as used for imaging. In other examples, the proton may be used only for therapy or for imaging.

The material of the detector apparatus 504 may be used to stop the proton. By stopping the proton, an energy the proton had when exiting the patient may be determined. For example, a depth that the particle travels into the material may depend on the stopping power of the material and the energy of the particle. This relationship may be given by: E_out1 minus E_in1 equals delta_E1. Delta_E1 may represent the exit energy when the proton exited the patient.

For multiple proton beamlets, scanned across the patient in a grid, there are multiple integral stopping powers (e.g., ∫_{stopping power}). Multiple proton beamlets may be measured with the integral stopping powers for each point on the grid. This measurement may be used to create a beam-eye-view image (e.g., an image with paths of protons through the patient, which may include a proton or may be overlaid on a cone beam CT).

In some examples, the proton beam 502 and the detector apparatus 504 may be affixed to a gantry. The gantry may rotate the proton beam 502 and the detector apparatus 504 about the patient, and collect projection images (e.g., of integral stopping power), throughout the movement. A proton may be created, such as with values corresponding to stopping powers of each volume element in the patient.

In an example, the detector apparatus 504 may include a cube or rectangular block of a material or materials. When using multiple materials, they may include respective multiple different stopping powers. For example, a sequence of materials with different stopping powers sp1, sp2, sp3, where sp1<sp2<sp3, may be used. The thickness of the material may be used to construct the layers.

In an example, the detector apparatus 504 may include a grid of orthogonal detectors (e.g., 64×64). The denser the material the fewer the number of detectors that may be used to measure a range of energies for a constant size detector, in some examples. When fewer detectors are used, the resolution of energy decreases, and uncertainty of the integral stopping power may increase. For example, the value of E_out may be higher with a smaller object, (e.g., pediatric patients), therefore a denser material may be used for a constant size image.

When the size of the individual element in the detector is a limiting factor, having a less dense material with more individual elements in the detector may allow for greater resolution in measuring the energy of the proton beamlet. The detector apparatus 504 may have orthogonal detectors to generate images using x-, y-, z-positions, or an angle of entrance of the proton beamlet with low uncertainty.

In an example, one technical problem that may be solved by the systems and techniques described herein is that in legacy detectors, energy is only be measured at the end of a proton path (e.g., when the detector stops the proton). For example, using large chunky detectors that only determine where the particle has stopped presents issues due to size constraints, lack of moveability, and inaccuracies. The systems and techniques described herein use the orthogonal detector arrays to measure energy at various points along a trajectory of the proton. This provides more accuracy, imaging capabilities, and the ability to determine a trajectory, which is not available to legacy detectors. Further, legacy devices may not be able to detect whether a proton beam has scattered or an angle of entry. Instead, these devices assume a straight-line path. The systems and techniques described herein provide the ability to detect a path based on the final position and at least one other position (e.g., entry to the detector apparatus 504 or exit from the patient). The systems and techniques described herein may determine an energy at the point of termination of the particle or along a trajectory of the particle.

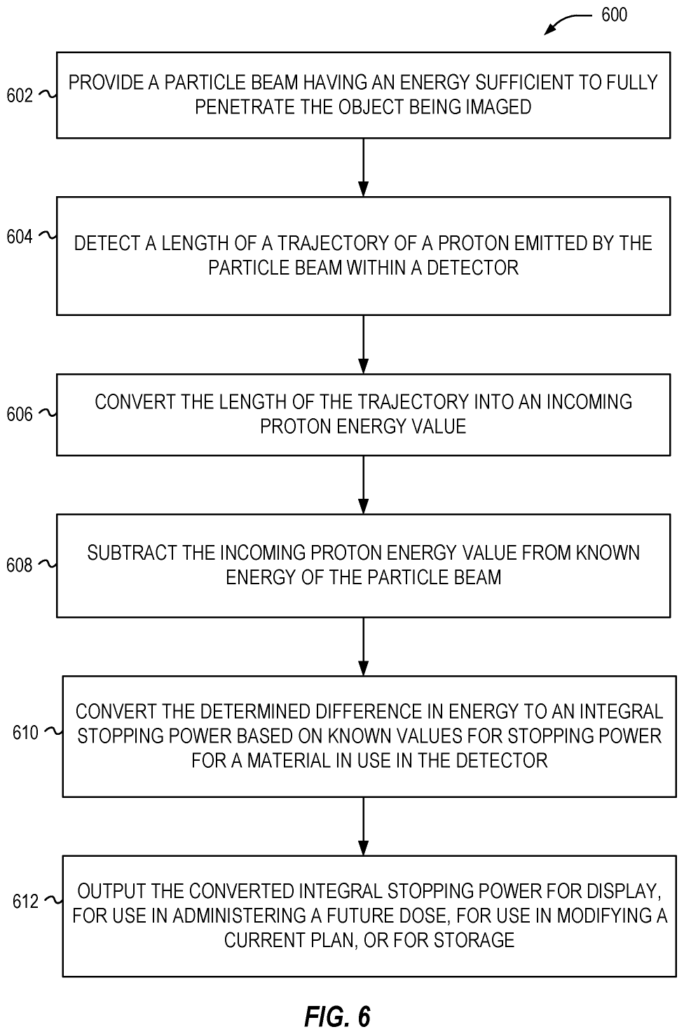

FIG. 6 illustrates a flowchart showing a technique 600 for delivering a plurality of particle beams from a rotating gantry towards a target, in accordance with an embodiment.

FIG. 6 illustrates a technique 600, including an operation 602 to provide a particle beam having an energy sufficient to fully penetrate the object being imaged.

The technique 600 includes an operation 604 to detect a length of a trajectory of a proton emitted by the particle beam within a detector.

The technique 600 includes an operation 606 to convert the length of the trajectory into an incoming proton energy value.

The technique 600 includes an operation 608 to subtract the incoming proton energy value from known energy of the particle beam.

The technique 600 includes an operation 610 to convert the determined difference in energy to an integral stopping power based on known values for stopping power for a material in use in the detector.

The technique 600 includes an operation 612 to output the converted integral stopping power for display, for use in administering a future dose, for use in modifying a current plan, or for storage.

In an example, the angle of entrance into the detector is determined based on the detected position of the beamlet on entry and the detected position of termination. In an example, scattering correction is applied based on the difference between the actual angle of entrance and the expected angle of entrance based on knowledge of the specific originating grid point of the beamlet. In an example, the particle beam includes a proton source and a detector, and wherein the proton source and the detector rotate about the object to be imaged. In an example, the set of acquired projections are reconstructed into a volumetric image of the stopping powers of the object being imaged.

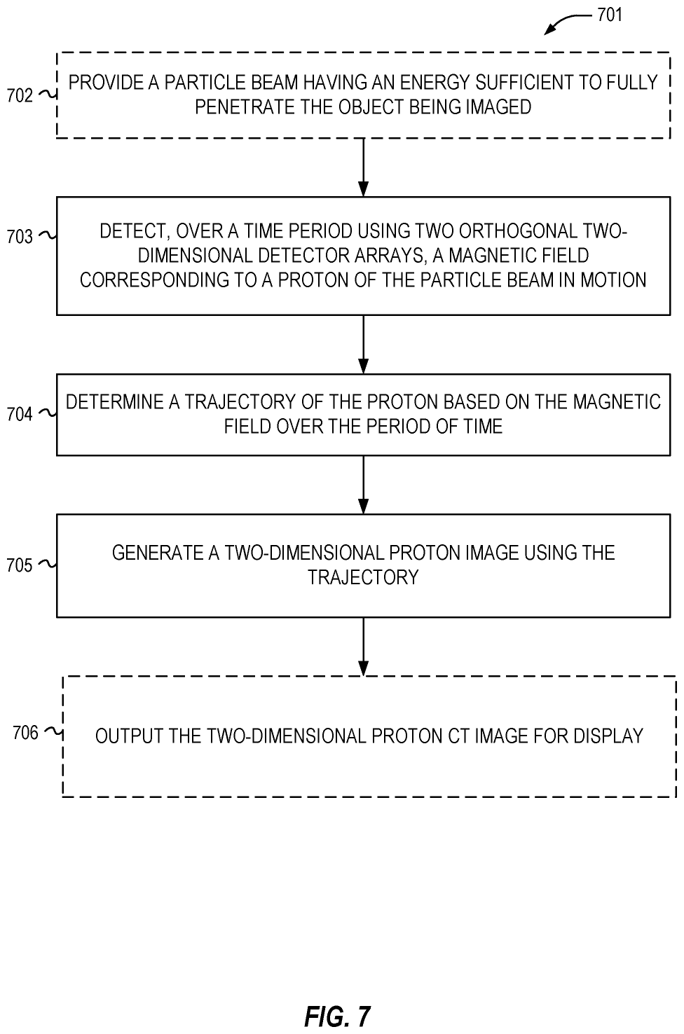

FIG. 7 illustrates a flowchart showing a technique 701 for particle reconstruction, such as in proton therapy, in accordance with an embodiment.

The technique 701 includes an optional operation 702 to provide a particle beam having an energy sufficient to fully penetrate an object (e.g., a patient, such as a portion of patient anatomy).

The technique 701 includes an operation 703 to detect, over a time period using two orthogonal two-dimensional detector arrays, a magnetic field corresponding to a proton of the particle beam in motion. The proton may be a treatment proton or a proton used for imaging or both. The particle beam may include a proton source, such as a proton beam. In an example, the proton beam and the two orthogonal two-dimensional detector arrays may be rotated about the object.

The technique 701 includes an operation 704 to determine a trajectory of the proton based on the magnetic field over the period of time. In some examples, the two orthogonal two-dimensional detector arrays are arranged such that detecting the magnetic field over the period of time includes detecting the magnetic field while the proton is within the object. In other examples, the two orthogonal two-dimensional detector arrays are arranged such that detecting the magnetic field over the period of time includes detecting the magnetic field after the proton has fully penetrated the object.

The technique 701 includes an operation 705 to generate a two-dimensional proton image using the trajectory. The two-dimensional proton image may include a proton image of the proton as it traversed the object.

The technique 701 includes an optional operation 706 to output the two-dimensional proton image for display. Operation 706 may include overlaying the two-dimensional proton image on a cone beam CT of the object.

The technique 701 may include applying scattering correction to the two-dimensional proton image, for example based on a difference between an actual angle of the trajectory and an expected angle of the trajectory (e.g., using an originating grid point of the particle beam).

In an example, the two orthogonal two-dimensional detector arrays are arranged in an apparatus. The apparatus may include a material configured to stop motion of the proton. In this example, the technique 701 may include detecting a distance traveled by the proton within the material until termination of the proton movement. The distance may be converted into an incoming proton energy value based on a stopping property of the material. The incoming proton energy value may be subtracted from a known initial energy of the proton to determine an amount of energy delivered to the object. In some versions of this example, the material is a poly(methyl methacrylate) (PMMA), such as Lucite or Plexiglass. In some versions of this example, the trajectory may be determined based on a detected position of the proton on entry into the material and a detected position of the proton at termination of the proton movement.

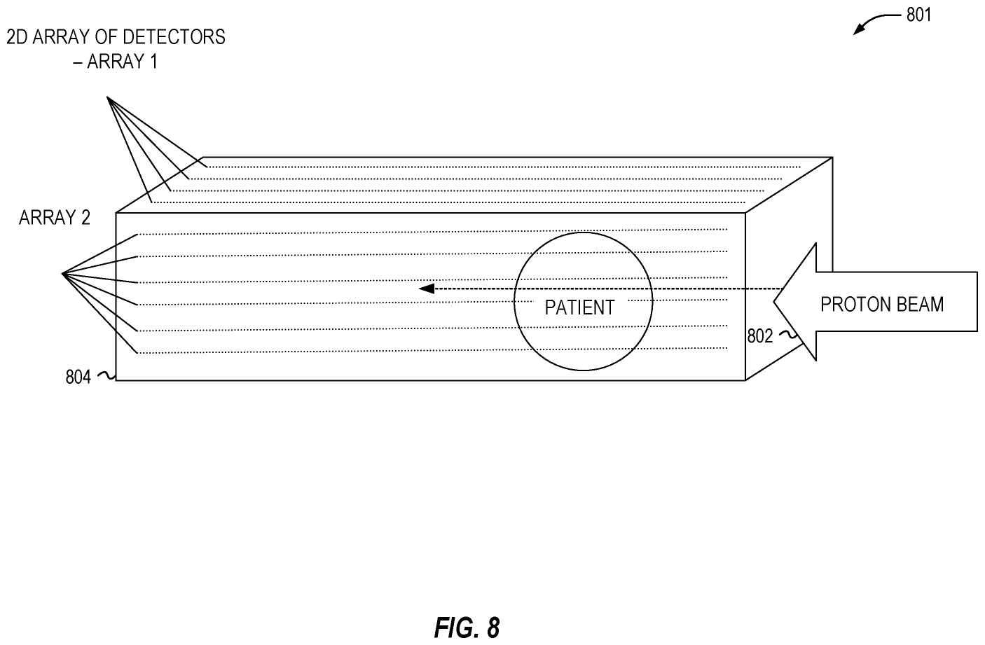

FIG. 8 illustrates a system 801 including a two orthogonal detector arrays, in accordance with an embodiment. The system 801 includes a proton beam 802 and a detector apparatus 804. While the detector apparatus 504 of FIG. 5 optional included a material, the detector apparatus 804 does not. Instead, the detector apparatus 804 is arranged such that the patient (e.g., a portion of patient anatomy, such as the head) may be placed within the detector apparatus 804. The detector apparatus 804 may use the two detector arrays to detect a proton while the proton is within the patient. The path of the proton may be tracked using the detector arrays to determine a trajectory of the proton within the patient. The proton may be directly tracked (e.g., compared to the indirect tracking of the trajectory described in FIG. 5) to produce an image of the trajectory of the proton through the patient. In some examples, the detector apparatus 804 may include a material to stop the proton in a portion of the detector apparatus 804, for example after the proton has exited the patient.

In some examples, the trajectory of a proton may be used for producing images of a stopping power. In these examples, the proton (integral) stopping power image is acquired (e.g., of the object/patient being imaged) with a distant (e.g., downstream relative to the patient) pair of detector arrays, which requires that the proton beamlets have energy sufficient to completely penetrate the object/patient. In an example, as a gantry rotates in a loop around incrementing gantry angles (or a rotating chair, with the beamline fixed and the distal detector array pair being fixed, in some examples) a Proton CT may be constructed (e.g., when the beam has energy sufficient to fully penetrate the patient).

In other examples, the trajectory may be used to obtain a full 3D trajectory or a terminating end point within a patient, such as when the proton beam does not have enough energy to pass through the patient (e.g., for the actual delivery). The full 3D trajectory may be used to provide a proton ray trace that may be used to reconstruct the actual dose delivered (e.g., by incorporating the actual trajectory and end point information, which may be compared to the simulated values used in treatment planning). These examples may include not acquiring a proton (e.g., integral) stopping power image, and instead using an orthogonal pair of detector arrays proximal to the head or other anatomy of the patient. In this example, there may not be a substance for detection, instead the anatomy patient stops the proton. This is the case where the proton beam does not have enough energy to pass through, and energies may be chosen specifically so that they do not pass through (and instead stop so that there is no exit dose past the edge of the tumor). The data in these examples may be used to reconstruct the dose (e.g., in conjunction with RT Ion Beams Treatment Record, which may have an accurate or substantially accurate measure of the meterset, such as number of protons delivered) to a given planned spot.

Each of the non-limiting examples described in this document may stand on its own, or may be combined in various permutations or combinations with one or more of the other examples.

Example 1 is a method of delivering a grid like pattern of particle beamlets through an object and to a detector, the method comprising: providing a particle beam having an energy sufficient to fully penetrate the object being imaged; where the length of the trajectory within the detector to the termination of the proton beamlet is detected; the length of the trajectory is converted in to an incoming proton energy value; the incoming proton energy value is subtracted from the known energy of the particle beam; the determined difference in energy in is converted to an integral stopping power based on the known values for stopping power for the material in use in the detector using a lookup table.

In Example 2, the subject matter of Example 1 includes, wherein the angle of entrance into the detector is determined based on the detected position of the beamlet on entry and the detected position of termination.

In Example 3, the subject matter of Examples 1-2 includes, wherein scattering correction is applied based on the difference between the actual angle of entrance and the expected angle of entrance based on knowledge of the specific originating grid point of the beamlet.

In Example 4, the subject matter of Examples 1-3 includes, wherein the particle beam includes a proton source and a detector, and wherein the proton source and the detector rotate about the object to be imaged.

In Example 5, the subject matter of Example 4 includes, where the set of acquired projections are reconstructed into a volumetric image of the stopping powers of the object being imaged.

Example 6 is a method of particle reconstruction, the method comprising: providing a particle beam having an energy sufficient to fully penetrate the object being imaged; detecting, over a time period using two orthogonal two-dimensional detector arrays, a magnetic field corresponding to a proton of the particle beam in motion; determining a trajectory of the proton based on the magnetic field over the period of time; generating a two-dimensional proton image using the trajectory; and outputting the two-dimensional proton image for display.

In Example 7, the subject matter of Example 6 includes, wherein the two-dimensional proton image is a proton image of the proton as it traversed the object.

In Example 8, the subject matter of Examples 6-7 includes, wherein the proton is a treatment proton, and wherein the object is a tumor.

In Example 9, the subject matter of Examples 6-8 includes, wherein outputting the two-dimensional proton image for display includes overlaying the two-dimensional proton image on a cone beam CT of the object.

In Example 10, the subject matter of Examples 6-9 includes, applying scattering correction to the two-dimensional proton image based on a difference between an actual angle of the trajectory and an expected angle of the trajectory using an originating grid point of the particle beam.

In Example 11, the subject matter of Examples 6-10 includes, wherein the particle beam includes a proton source, and wherein the proton source and the two orthogonal two-dimensional detector arrays rotate about the object.

In Example 12, the subject matter of Examples 6-11 includes, wherein the two orthogonal two-dimensional detector arrays are arranged in an apparatus, the apparatus including a material configured to stop motion of the proton, and further comprising: detecting a distance traveled by the proton within the material until termination of the proton movement; converting the distance into an incoming proton energy value based on a stopping property of the material; and subtracting the incoming proton energy value from a known initial energy of the proton to determine an amount of energy delivered to the object.

In Example 13, the subject matter of Example 12 includes, wherein the material is a Poly(methyl methacrylate) (PMMA).

In Example 14, the subject matter of Examples 12-13 includes, wherein the trajectory is determined based on a detected position of the proton on entry into the material and a detected position of the proton at termination of the proton movement.

In Example 15, the subject matter of Examples 6-14 includes, wherein the two orthogonal two-dimensional detector arrays are arranged such that detecting the magnetic field over the period of time includes detecting the magnetic field while the proton is within the object.

In Example 16, the subject matter of Examples 6-15 includes, wherein the two orthogonal two-dimensional detector arrays are arranged such that detecting the magnetic field over the period of time includes detecting the magnetic field after the proton has fully penetrated the object.

Example 17 is a system comprising: a proton beam to provide a particle beam having an energy sufficient to fully penetrate an object being imaged; two orthogonal two-dimensional detector arrays to detect, over a time period, a magnetic field corresponding to a proton of the particle beam in motion; a processor; and memory including instructions, which when executed by the processor, cause the processor to: determine a trajectory of the proton based on the magnetic field over the period of time; generate a two-dimensional proton image using the trajectory; and output the two-dimensional proton image for display.

In Example 18, the subject matter of Example 17 includes, wherein the two-dimensional proton image is a proton image of the proton as it traversed the object.

In Example 19, the subject matter of Examples 17-18 includes, wherein the proton beam includes a proton source, and further comprising a gantry configured to cause the proton source and the two orthogonal two-dimensional detector arrays to rotate about the object.

In Example 20, the subject matter of Examples 17-19 includes, wherein the two orthogonal two-dimensional detector arrays are arranged in an apparatus, the apparatus including a material configured to stop motion of the proton, and wherein the instructions further include operations to cause the processor to: detect a distance traveled by the proton within the material until termination of the proton movement; convert the distance into an incoming proton energy value based on a stopping property of the material; and subtract the incoming proton energy value from a known initial energy of the proton to determine an amount of energy delivered to the object.

In Example 21, the subject matter of Example 20 includes, wherein the material is a Poly(methyl methacrylate) (PMMA).

In Example 22, the subject matter of Examples 20-21 includes, wherein the trajectory is determined based on a detected position of the proton on entry into the material and a detected position of the proton at termination of the proton movement.

In Example 23, the subject matter of Examples 17-22 includes, wherein the two orthogonal two-dimensional detector arrays are arranged such that detecting the magnetic field over the period of time includes detecting the magnetic field while the proton is within the object.

In Example 24, the subject matter of Examples 17-23 includes, wherein the two orthogonal two-dimensional detector arrays are arranged such that detecting the magnetic field over the period of time includes detecting the magnetic field after the proton has fully penetrated the object.

Example 25 is at least one machine-readable medium including instructions for performing particle reconstruction, which when executed by processing circuitry, cause the processing circuitry to: detect, over a time period using two orthogonal two-dimensional detector arrays, a magnetic field corresponding to a proton in motion, the proton delivered by a particle beam to an object; determine a trajectory of the proton based on the magnetic field over the period of time; generate a two-dimensional proton image using the trajectory; and output the two-dimensional proton image for display.

Example 26 is at least one machine-readable medium including instructions that, when executed by processing circuitry, cause the processing circuitry to perform operations to implement of any of Examples 1-25.

Example 27 is an apparatus comprising means to implement of any of Examples 1-25.

Example 28 is a system to implement of any of Examples 1-25.

Example 29 is a method to implement of any of Examples 1-25.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

1. A method of particle reconstruction, the method comprising:
providing a particle beam having an energy sufficient to fully penetrate the object being imaged;
detecting, over a time period using two orthogonal 2D detector arrays, a magnetic field corresponding to a proton of the particle beam in motion;
determining a trajectory of the proton based on the magnetic field over the period of time;
generating a 2D proton image using the trajectory; and
outputting the 2D proton image for display.

2. The method of claim 1, wherein the 2D proton image is a proton image of the proton as it traversed the object.

3. The method of claim 1, wherein the proton is a treatment proton, and wherein the object is a tumor.

4. The method of claim 1, wherein outputting the 2D proton image for display includes overlaying the 2D proton image on a cone beam CT of the object.

5. The method of claim 1, further comprising applying scattering correction to the 2D proton image based on a difference between an actual angle of the trajectory and an expected angle of the trajectory using an originating grid point of the particle beam.

6. The method of claim 1, wherein the particle beam includes a proton source, and wherein the proton source and the two orthogonal 2D detector arrays rotate about the object.

7. The method of claim 1, wherein the two orthogonal 2D detector arrays are arranged in an apparatus, the apparatus including a material configured to stop motion of the proton, and further comprising:
detecting a distance traveled by the proton within the material until termination of the proton movement;
converting the distance into an incoming proton energy value based on a stopping property of the material; and
subtracting the incoming proton energy value from a known initial energy of the proton to determine an amount of energy delivered to the object.

8. The method of claim 7, wherein the material is a Poly(methyl methacrylate) (PMMA).

9. The method of claim 7, wherein the trajectory is determined based on a detected position of the proton on entry into the material and a detected position of the proton at termination of the proton movement.

10. The method of claim 1, wherein the two orthogonal 2D detector arrays are arranged such that detecting the magnetic field over the period of time includes detecting the magnetic field while the proton is within the object.

11. The method of claim 1, wherein the two orthogonal 2D detector arrays are arranged such that detecting the magnetic field over the period of time includes detecting the magnetic field after the proton has fully penetrated the object.

12. A system comprising:
a proton beam to provide a particle beam having an energy sufficient to fully penetrate an object being imaged;
two orthogonal 2D detector arrays to detect, over a time period, a magnetic field corresponding to a proton of the particle beam in motion;
a processor; and
memory including instructions, which when executed by the processor, cause the processor to:
determine a trajectory of the proton based on the magnetic field over the period of time;
generate a 2D proton image using the trajectory; and
output the 2D proton image for display.

13. The system of claim 12, wherein the 2D proton image is a proton image of the proton as it traversed the object.

14. The system of claim 12, wherein the proton is a treatment proton, and wherein the object is a tumor.

15. The system of claim 12, wherein the proton beam includes a proton source, and further comprising a gantry configured to cause the proton source and the two orthogonal 2D detector arrays to rotate about the object.

16. The system of claim 12, wherein the two orthogonal 2D detector arrays are arranged in an apparatus, the apparatus including a material configured to stop motion of the proton, and wherein the instructions further include operations to cause the processor to:
detect a distance traveled by the proton within the material until termination of the proton movement;
convert the distance into an incoming proton energy value based on a stopping property of the material; and
subtract the incoming proton energy value from a known initial energy of the proton to determine an amount of energy delivered to the object.

17. The system of claim 16, wherein the material is a Poly(methyl methacrylate) (PMMA).

18. The system of claim 16, wherein the trajectory is determined based on a detected position of the proton on entry into the material and a detected position of the proton at termination of the proton movement.

19. The system of claim 12, wherein the two orthogonal 2D detector arrays are arranged such that detecting the magnetic field over the period of time includes detecting the magnetic field while the proton is within the object.

20. The system of claim 12, wherein the two orthogonal 2D detector arrays are arranged such that detecting the magnetic field over the period of time includes detecting the magnetic field after the proton has fully penetrated the object.

21. At least one machine-readable medium including instructions for performing particle reconstruction, which when executed by processing circuitry, cause the processing circuitry to:
detect, over a time period using two orthogonal 2D detector arrays, a magnetic field corresponding to a proton in motion, the proton delivered by a particle beam to an object;
determine a trajectory of the proton based on the magnetic field over the period of time;
generate a 2D proton image using the trajectory; and
output the 2D proton image for display.
